(19) United States
(12) Patent Application Publication
(10) Pub. No.: US 2023/0051579 A1
(43) Pub. Date: Feb. 16, 2023

(54) PROCESS FOR PREPARING CANNABINOID-CONTAINING PARTICLES

(71) Applicant: STABICAN B.V., Almere (NL)

(72) Inventor: Robin DE BRUIJN, Eindhoven (NL)

(21) Appl. No.: 17/792,088

(22) PCT Filed: Jan. 8, 2021
(86) PCT No.: PCT/NL2021/050011

§ 371 Date: Jul. 11, 2022

(30) Foreign Application Priority Data
Jan. 10, 2020 (NL) 2024652

(51) Int. Cl. A61K 9/50; A61K 31/352

(52) U.S. Cl. CPC A61K 9/5089; A61K 31/352; A61K 9/5015

(57) ABSTRACT

The invention relates to process for preparing cannabinoid-containing particles, wherein first a solution is provided wherein the solvent has a water solubility in the range of 2-100 g/L at 25° C. and wherein the solution comprises 1) a cannabis component comprising THC; and 2) a shell-forming component comprising one or more compounds selected from the group of C10-C30 fatty alcohols, C10-C30 fatty acids and esters of C10-C30 fatty alcohols and C10-C30 fatty acids. Then, droplets of the solution are generated in an aqueous medium and the solvent is allowed to migrate from the droplets to the aqueous medium to thereby form solid particles wherein a shell of shell-forming component encapsulates the cannabis component.

### FIELD OF THE INVENTION

The present invention relates to a process for preparing cannabinoid-containing particles, to a particle obtainable by such method and to a cannabinoid-containing particle.

### BACKGROUND

Cannabis plants produce a group of chemicals called cannabinoids, which are terpenophenolic compounds with a common structural motif. They differ mainly in the way their common precursor cannabigerol is cyclized. Cannabinoids in the form of cannabis and extracts thereof have been used for centuries for both medicinal and recreational purposes. They are attractive due to their particular psychoactive and physical effects.

More than hundred cannabinoids have been identified in Cannabis plants, of which the most prevalent are delta-9-tetrahydrocannabinol (THC), cannabidiol (CBD), and cannabinol (CBN). THC is the primary psychoactive component of the plant and has also found use in the treatment of a wide range of medical conditions. More specifically, THC binds to specific receptors in the brain called cannabinoid receptors and, in doing so, causes pain reduction, may reduce aggression, can stimulate appetite, and helps reduce nausea.

The widely known recreational use of cannabis involves the inhalation of smoke of burning cannabis plant parts, but many patients and other consumers of cannabis prefer to administer cannabis by eating or drinking it, rather than smoking. Moreover, especially for medical purposes, it is important that an exact dosage of one or more known cannabinoids, in particular THC, can be administered, which can hardly be achieved by smoking.

Oral dosage forms of cannabinoids are a logical alternative for smoking. However, despite many research and development efforts, the oral dosage forms known to date still suffer from serious shortcomings. It is difficult to prepare them in a reproducible way, in particular to include the same amounts of cannabinoid(s) in each dosage composition. Further, the stability of known dosage forms is often insufficient, which is in particular encountered when it is aimed to use the composition as a pharmaceutical base product for the preparation of various kinds cannabinoid-based medicine since this requires long shelf lives. Another disadvantage is that conventional dosage forms as a substance are difficult to handle, which is in particular caused by their tackyness. This property is mainly due to the oily nature of THC, which results in a high tackiness of compositions that are made from it. Especially microparticles and nanoparticles wherein THC is present in a filler material (e.g. a wax) suffer from tackiness, which does not allow the manufacture of oral dosage forms in the form of a powder.

For example, WO2016/144376A1 describes dispersions of phospholipids and cannabinoids, allegedly present as nanoparticles, that are in a liquid or gel form and are therefore tacky or at least far remote from being a dry solid. Moreover, since the particles are characterized as dynamic structures wherein cannabinoids are localized in and on an outer membrane, there is no true and durable encapsulation of a cannabinoid. In this setting, the cannabinoids are in fact exposed to the surrounding atmosphere of the material, which is obviously limiting to the long-term stability of the cannabinoids.

A particular type of cannabinoid dosage form concerns those comprising the so-called whole-plant cannabis extracts. Such mixture of many known and unknown components is interesting from a medicinal point of view, but the reproducible preparation of stable and easy-to-handle formulations thereof remains a big challenge.

It can be generally stated that at present, dosages of THC are not available in a satisfactory form, e.g. a form that is non-tacky and/or has a long shelf-life. In particular, there is a need for a standardized formulation comprising THC, preferably in a powder form, as a way to enable consumers of cannabis to accurately and repeatably deliver the same dose of THC to address their (medical) needs. More in particular, there is a need for a pharmaceutical base product that can serve as a standard for the preparation of cannabinoid-based medicine.

### SUMMARY OF THE INVENTION

It is therefore an object of the present invention to provide a formulation comprising THC that does not suffer from one or more of the abovementioned shortcomings.

It has now been found that a particular encapsulation method may overcome these shortcomings.

Accordingly, the invention relates to a process for preparing cannabinoid-containing particles, comprising
providing a solution wherein the following components are dissolved in a solvent that has a water solubility in the range of 2-100 g/L at 25° C.:
   a cannabis component comprising delta-9-tetrahydrocannabinol;
   a shell-forming component comprising one or more compounds selected from the group of C10-C30 fatty alcohols, C10-C30 fatty acids and esters of C10-C30 fatty alcohols and C10-C30 fatty acids;
generating droplets of the solution in an aqueous medium and allowing the solvent to migrate from the droplets to the aqueous medium to thereby form solid particles wherein a shell of shell-forming component encapsulates the cannabis component.

### BRIEF DESCRIPTION OF THE DRAWINGS

FIG. 1 displays a micrograph of THC-containing particles that are produced according to a method according to the invention.

### DETAILED DESCRIPTION OF THE INVENTION

The solvent has a solubility in water that is in the range of 2-100 g/L at 25° C. This range on the one hand ensures that the solution of the cannabis component and the shell-forming component in the solvent is capable of existing as droplets in a water phase; and on the other hand that the solvent migrates from the droplet to the water phase. For example, a solvent with a solubility higher than 100 g/L (including infinite solubility), will not form droplets, or only droplets with insufficient stability, so that the two water-insoluble components do not phase separate and solid particles are not formed. Also, a solvent with a solubility lower than 2 g/L (including complete insolubility) will nevertheless form droplets, but its migration from the droplets to the water phase, if any, cannot be accomplished under satisfying conditions.

The solubility of the solvent in water may also be in the range of 6-90 g/L at 25° C., in the range of 8-80 g/L at 25° C., in the range of 10-70 g/L at 25° C., in the range of 12-60 g/L at 25° C., or in the range of 15-50 g/L at 25° C. It may also be in the range of 5-75 g/L at 25° C., in the range of 8-50 g/L at 25° C. or in the range of 10-40 g/L at 25° C.

The solvent may be selected from the group of benzyl alcohol, 1-butanol, n-butyl acetate, gamma-butyrolacton, chloroform, 1,2-dichloroethane, diethylene glycol, diethyl ether, diethoxyethane, di-isopropylether, ethyl acetate, methyl t-butyl ether, methylene chloride, N-methyl-2-pyrrolidinone, nitromethane, 1-pentanol, 2-pentanol, 3-pentanol, 3-pentanone, benzaldehyde, prenol, o-cresol, m-cresol, and p-cresol. The solvent may also be a terpenoid with a water solubility in the range of 2-100 g/L at 25° C. In particular, the solvent is benzyl alcohol or methylene chloride.

The solvent usually has a molar mass of less than 200 g/mol. It is in particular less than 150 g/mol. It may also be less than 140 g/mol, less than 125 g/mol or less than 100 g/mol.

The solution in a process of the invention comprises a homogeneous mixture of the two components that in the end make up the cannabinoid-containing particles. The migration of the solvent from the droplet to the aqueous medium has the effect that both components come out of solution and gain their natural appearance; the C10-C30 fatty acids, alcohols and ester are solid (or at least waxy), while the THC (eventually accompanied by other cannabinoids) is a liquid or an oil. Moreover, the solvent migration also results in the separation of both components, wherein the cannabis component constitutes the inner part of the particles and the shell-forming component constitutes a shell that completely surrounds the cannabis component.

Usually, the solution consists of these two components, i.e. no other dissolved or undissolved substances are present in the solution. It is however possible that certain additives are contained in the solution, which either end up in the produced particle or migrate together with the solvent. For example, active pharmaceutical ingredients other than cannabinoids may be present. Also, other extractives from the Cannabis plant may be present, especially when the cannabis component comprises a whole-plant cannabis extract. It is also possible that a co-solvent is present in the solution, which migrates together with the solvent (i.e. the primary solvent) out of the droplet (or particle) into the aqueous medium. For example, such co-solvent is a solvent selected from the group of the (primary) solvents mentioned above.

In the solution, the weight ratio of cannabinoid component to shell-forming component is usually in the range of 0.05:0.95 to 0.95:0.05. The lower this ratio, the thicker the shell and the less cannabinoid is contained by the shell (in relative terms). Migration of the solvent is likely accompanied by the migration of minor amounts of cannabinoid that subsequently may get stuck in the shell during formation of the particles. When the particles indeed contain a minor but significant amount of delta-9-tetrahydrocannabinol in their shell, they become more tacky due to the tacky nature of delta-9-tetrahydrocannabinol. It appeared that the tackiness of the particles can be decreased by decreasing the weight ratio of cannabinoid component to shell-forming component in the solution. It is therefore advantageous when the weight ratio of cannabinoid component to shell-forming component, in particular of delta-9-tetrahydrocannabinol to shell-forming component, in the solution is low. On the other hand, however, such low ratio is unfavorable for the weight percentage of cannabinoid in the final particle, and thus also in the entire formulation. From this point of view, it is generally preferred that the cannabinoid weight percentage in a formulation is as high as possible. Therefore, the weight ratio of cannabinoid component to shell-forming component in the solution in a process of the invention is a balance between tackiness and cannabinoid content of the product. Preferably, therefore, the ratio is in the range of 0.25:0.75 to 0.95:0.05, in particular in the range of 0.50:0.50 to 0.90:0.10. It may also be in the range of 0.40:0.60 to 0.80:0.20, or in the range of 0.60:0.40 to 0.95:0.05.

Thus, the tackiness of the particles can be tuned by varying the weight ratio of cannabinoid component to shell-forming component in the solution, in particular the weight ratio of delta-9-tetrahydrocannabinol to shell-forming component. It appeared possible to prepare particles in suspension (in particular a suspension in water) that are not tacky at all. Generally, this is achieved when the shell-forming component is present in an amount of at least 30 wt. % (weight ratio of cannabinoid component to shell-forming component in the solution is at least 30:70). This allows the preparation of a non-tacky delta-9-tetrahydrocannabinol formulation. This is unprecedented in the art, since no solution has yet been found to the problem of tackiness—a problem that has dominated the field of cannabinoid encapsulation for decades. In addition, the cannabionoids that are contained in the particles are surprisingly stable. The low tackiness and the high stability allow the preparation of a delta-9-tetrahydrocannabinol-containing pharmaceutical base product, which is a long-felt need in the medical field.

With only a minor amount of cannabinoid incorporated in the material of the shell itself (or none at all), the interior of the shell (i.e. the encapsulated area) should of course contain the majority (or all) of the cannabinoid. To confirm this, the amount of cannabinoid that is present in the product of the process of the invention was determined, from which the fraction of initially present cannabinoid be calculated that has been encapsulated by the process (encapsulation efficiency). This was for example performed for a process wherein THC was the only cannabinoid present and wherein benzyl alcohol was used as the solvent (as is further elaborated in the Examples' section 4). It appeared that it is possible to encapsulate 85 wt. % of the THC that was added in the process, and that the resulting product was a dry, non-tacky solid.

The cannabis component consists of one or more cannabinoids, and comprises at least delta-9-tetrahydrocannabinol. The cannabis component may consist of delta-9-tetrahydrocannabinol, but it is also possible that one or more cannabinoids other than delta-9-tetrahydrocannabinol are present. For example, the cannabis component comprises one or more cannabinoids selected from the group of cannabidiol, cannabinol and cannabigerol. For example, at least 95 wt. % of the cannabis component consists of delta tetrahydrocannabinol and one or more cannabinoids selected from the group of cannabidiol, cannabinol and cannabigerol (the remaining 5 wt. % then consists of other cannabinoids). In particular, at least 98 wt. %, more in particular at least 99 wt. % and even more in particular at least 99.5 wt. % of the cannabis component consists of delta-9-tetrahydrocannabinol and one or more cannabinoids selected from the group of cannabidiol, cannabinol, cannabigerol, delta9-tetrahydrocannabinolic acid, and cannabidiolic acid.

The delta-9-tetrahydrocannabinol is usually the main cannabionoid present in the cannabis component, i.e. more than 50 wt. % of the cannabis component consists of delta-9-tetrahydrocannabinol. For example, the content of delta-9-tetrahydrocannabinol is at least 60 wt. %, at least 75 wt. %, at least 85 wt. % or at least 90 wt. %. In particular, the content is at least 95 wt. %. More in particular, at least 99 wt. %, even more in particular at least 99.5 wt. %, yet even more in particular at least 99.9 wt. % of the cannabis component consists of delta-9-tetrahydrocannabinol.

In a method of the invention, the one or more cannabinoids in the solution may be obtained directly or indirectly by extraction from a Cannabis plant (e.g. delta-9-tetrahydrocannabinol may be obtained after extraction by decarboxylation of extracted tetrahydrocannabinolic acid). One or more particular cannabinoids may be isolated from the extract and then be used in the cannabis component in the solution. Alternatively, the entire extract may be used in the solution. In such case, the cannabis component comprises a so-called whole-plant cannabis extract.

The C10-C30 fatty alcohol may be an alcohol selected from the group of capric alcohol, lauryl alcohol, myristyl alcohol, cetyl alcohol, palmitoleyl alcohol, stearyl alcohol, isostearyl alcohol, oleyl alcohol, elaidyl alcohol, petroselinyl alcohol, elaeostearyl alcohol, arachyl alcohol, gadoleyl alcohol, behenyl alcohol, erucyl alcohol and brassidyl alcohol.

The C10-C30 fatty acid may be an acid selected from the group of capric acid, lauric acid, palmitic acid, palmitoleic acid, stearic acid, isostearic acid, oleic acid, elaidic acid, petroselic acid, elaeostearic acid, arachic acid, gadoleic acid, behenic acid and erucic acid.

In particular, the shell-forming component in the solvent comprises cetyl alcohol and/or cetyl palmitate.

The temperature at which the process is performed (i.e. the operating temperature) may in principle be any temperature above the freezing point of water and below the boiling point of the solvent. For example, the temperature may be in the range 0-60° C., in the range of 5-50° C. or in the range of 10-35° C. Usually, however, the temperature is in the range of 15-30° C. In particular, it is in the range of 20-25° C.

The invention further relates to a cannabinoid-containing particle obtainable by the process described above.

A cannabinoid-containing particle that is obtained by a process of the invention has a core of one or more cannabinoids including delta-9-tetrahydrocannabinol and a shell encapsulating this core. The shell compartimentalizes the one or more cannabinoids and so protects them against influences from the outer environment such as micro-organisms or reactive compounds such as oxygen. Accordingly, the invention further relates to a cannabinoid-containing particle, wherein
one or more cannabinoids are encapsulated by a shell of one or more compounds selected from the group of C10-C30 fatty alcohols, C10-C30 fatty acids and esters of C10-C30 fatty alcohols and C10-C30 fatty acids;
the content of cannabinoid in a particle is in the range of 5-95 wt. %, in particular in the range of 25-85 wt. %.

Usually, the content of cannabinoid in a particle of the invention is in the range of 20-80 wt. %, preferably it is in the range of 25-75 wt. %, more preferably it is in the range of 30-70 wt. % (the weight percentages are based on the total weight of the particle). Assuming no other constituents are present (in particular not encapsulated), it then follows that the shell constitutes 5-95 wt. % of the particle, in particular 15-75 wt. %. Usually, it is 20-80 wt. %, preferably 25-75 wt. %, more preferably 30-70 wt. %.

As noted earlier, a low or absent tackiness is likely the result of a shell that contains only small amounts of cannabinoid(s), or none at all, respectively. For example, particles with a THC content of 71 wt. % (and a shell content of 29 wt. %) were prepared (see Examples' section 5). It was found that this product was dry and not tacky. This is an indication that the shell (i.e. the substance of the shell itself) of a particle of the invention is substantially free of cannabinoid, in particular of delta-9-tetrahydrocannabinol. This means that the presence of cannabinoid in the particle is limited to the cannabinoid core.

This is in contrast to many known cannabinoid-containing particles wherein cannabinoid is enclosed on the one hand, but on the other hand forms part of the enclosing material or is even localized onto such material (i.e. at the exterior of a particle at the interface with a surrounding atmosphere). This makes prior art particles sticky and the cannabinoid subject to deterioration. For example, WO2016/144376A1 describes particles of a dynamic nature, wherein cannabinoids reside not only on their inside, but also on their outside.

In similar disclosures of cannabinoid-containing particles, a wax is present as a filler material that forms an interpenetrating network between the cannabinoid(s). In particles with such composition, a plurality of cannabinoid domains is present. The wax then remains tacky due to such morphology. In contrast, a particle of the invention in principle comprises one domain, namely the core of the particle.

It has been demonstrated that the cannabinoid in a particle of the invention is stable during a period of at least 13 months, which is (beside the non-tackyness) also an effect of the excellent shielding that is provided by the process of the invention.

In a particle of the invention, the weight ratio of cannabinoid to shell is typically in the range of 0.25:0.75 to 0.95:0.05. It may also be 0.50:0.50 to 0.90:0.10, in the range of 0.40:0.60 to 0.80:0.20, or in the range of 0.60:0.40 to 0.95:0.05. This ratio is reflected by the ratio of cannabis component to shell-forming component in the solution that is used for preparing the particle.

As demonstrated in the Examples' section 5, particles have been prepared having a THC content of 71 wt. %. Not only is this value surprising, but it is also surprising also that this has been achieved by losing only 15 wt. % of the initially present THC (i.e. the THC present at the start of the encapsulation procedure). In encapsulations in general, it is often seen that in order to achieve a high ratio of encapsulated compound to encapsulant in the product, the initial ratio of both components has to be much higher to account for the many losses during the encapsulation process. In the present invention, however, the encapsulation of high amounts of cannabinoid does not go hand in hand with large losses of cannabinoid. Thus, it is an advantage of the present invention that a high THC weight percentage in the particles is realized in combination with a high encapsulation efficiency.

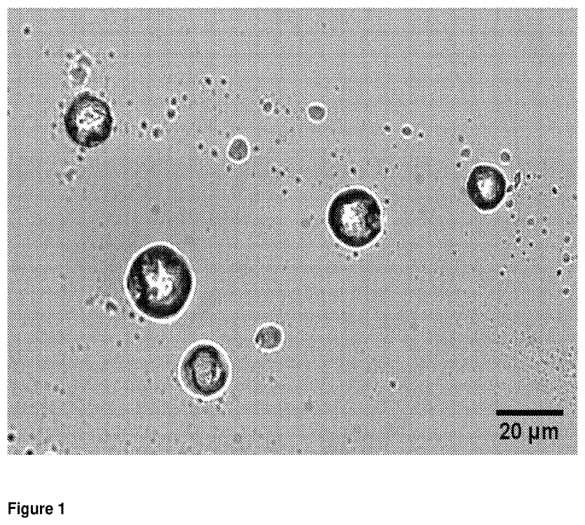

THC-containing particles of the invention are displayed in the micrograph of FIG. 1, recorded with an optical microscope. They are more or less spherical with a diameter of 10-20 μm. Their outer surface appears not completely smooth, as it appears to be crumpled a little bit. The displayed particles are in an ambient atmosphere and do not stick to one another.

A cannabinoid-containing particle of the invention is usually globular. This shape is governed by the shape of the initial droplet in the process of the invention, which is usually globular.

The diameter of a particle of the invention is usually in the range of 100 nm-500 μm, in particular in the range of 250 nm-400 μm, more in particular in the range of 1-250 μm, even more in particular in the range of 3-100 μm or yet even more in particular in the range of 5-50 μm. For example, it is 200 nm or more, 300 nm or more, 400 nm or more, 500 nm or more, 750 nm or more, 1 μm or more, 2 μm or more, 3 μm or more, 4 μm or more, 5 μm or more, 6 μm or more, 7 μm or more, 8 μm or more, 9 μm or more, 10 μm or more, 12 μm or more, 14 μm or more, 20 μm or more, 30 μm or more, 40 μm or more, 50 μm or more, 75 μm or more, 100 μm or more, 200 μm or more, 300 μm or more, or 400 μm or more. It may also be 500 μm or less, 400 μm or less, 300 μm or less, 200 μm or less, 100 μm or less, 75 μm or less, 70 μm or less, 25 μm or less, 20 μm or less, 15 μm or less, 10 μm or less, 8 μm or less, 7 μm or less, 6 μm or less, 5 μm or less, 4 μm or less, 3 μm or less, 2 μm or less, 1 μm or less, 800 nm or less, 600 nm or less, 400 nm or less or 200 nm or less.

Many medical indications or uses have been identified for cannabinoids, which has resulted in numerous applications of cannabinoids in the medical field. Accordingly, the invention further relates to a pharmaceutical composition comprising a cannabinoid-containing particle as described above and an excipient.

The term excipient as used herein refers to any substance, generally pharmaceutically inert, used to formulate active pharmaceutical ingredients (API) into pharmaceutical formulations. For example, an excipient is selected from the group of diluents, binders, glidants, lubricants, colouring agents and disintegrants. The excipient material itself may be composed of one or more materials selected from the group of sugar alcohols, polyols (e.g. sorbitol, mannitol, xylitol), crystalline sugars, monosaccharides (e.g. glucose, arabinose), disaccharides (e.g. maltose, saccharose, dextrose, lactose), oligosaccharides (e.g. dextrins, cyclodextrins), polysaccharides (e.g. cellulose starch and derivatives thereof), inorganic salts (e.g. sodium chloride, calcium carbonate, magnesium carbonate, talc), and organic salts (e.g. sodium lactate, magnesium stearate).

The invention further relates to a cannabinoid-containing particle or a pharmaceutical composition as described above for use as a medicament.

The medical indications or uses for cannabinoids can broadly be broken down into the following categories: anti-nauseant and appetite stimulant, anti-spasmodic and anti-convulsant, analgesic (pain reliever), anti-inflammatory and immune system modulator, anxiolytic (anxiety reliever) and anti-depressant for mood disorders, harm reduction substitute for alcohol, opiates, and other drugs.

More specifically, cannabinoids have been reported to exhibit a therapeutic effect in the treatment of allergies, inflammations, infections, asthma, arthritis, epilepsy, depression, migraine, psychotic behavior, bipolar disorders, anxiety disorder, drug dependency and withdrawal syndromes, glaucoma, AIDS wasting syndrome, neuropathic pain, spasticity associated with multiple sclerosis, fibromyalgia, (chemotherapy-induced) nausea, anorexia, multiple sclerosis, gastrointestinal motility disorders, irritable bowel syndrome, appetite disorders, cachexia, and cramps. Further, cannabinoids have been reported to be effective in the treatment of diabetes, cancer, osteoporosis, sleep apneu, phantom limb, spinal/brain injury, Alzheimer's disease, glaucoma, neurodegenerative disease, Parkinson's disease, seizure, fatigue, post traumatic stress disorder, stress, organ rejection, ischemia, psoriasis, bone defects, AIDS, immunosuppressive disorder, muscular dystrophy, and vascular disorders.

Accordingly, the invention further relates to a cannabinoid-containing particle or a pharmaceutical composition as described above for use in the treatment of any of these conditions.

The invention further relates to a method for treating a medical condition of a human or an animal, comprising administering to the human or animal an effective amount of a cannabinoid-containing particle or a pharmaceutical composition as described above, wherein the medical condition is selected from the group of allergies, inflammations, infections, asthma, arthritis, epilepsy, depression, migraine, psychotic behavior, bipolar disorders, anxiety disorder, drug dependency and withdrawal syndromes, glaucoma, AIDS wasting syndrome, neuropathic pain, spasticity associated with multiple sclerosis, fibromyalgia, (chemotherapy-induced) nausea, anorexia, multiple sclerosis, gastrointestinal motility disorders, irritable bowel syndrome, appetite disorders, cachexia, and cramps.

As used herein, the term “effective amount” means that amount of a drug or pharmaceutical agent that will elicit the biological or medical response of an animal or human that is being sought, for instance, by a researcher or clinician.

The invention further relates to the use of a cannabinoid-containing particle or a pharmaceutical composition as described above for treating a human or an animal.

The invention further relates to the use of a cannabinoid-containing particle or a pharmaceutical composition as described above for the manufacture of a medicament for the treatment of a medical condition, wherein the medical condition is selected from the group of allergies, inflammations, infections, asthma, arthritis, epilepsy, depression, migraine, psychotic behavior, bipolar disorders, anxiety disorder, drug dependency and withdrawal syndromes, glaucoma, AIDS wasting syndrome, neuropathic pain, spasticity associated with multiple sclerosis, fibromyalgia, (chemotherapy-induced) nausea, anorexia, multiple sclerosis, gastrointestinal motility disorders, irritable bowel syndrome, appetite disorders, cachexia, and cramps.

The term medical condition as used herein is a broad term that includes all diseases, lesions, disorders, or nonpathologic condition that normally receives medical treatment, such as pregnancy or childbirth.

### EXAMPLES

1. Materials

The chemicals and solvents were purchased from commercial sources and used without further purification. If necessary, residual water was removed prior to use. When water was used, it was demineralized prior to use.

The following 10 mg/ml stock solutions were prepared:

1) cetyl alcohol in dichloromethane
2) cetyl palmitate in dichloromethane
3) THC in dichloromethane
4) cetyl alcohol in benzyl alcohol
5) cetyl palmitate in benzyl alcohol
6) THC in benzyl alcohol

The used homogenizer was an IKA T10 Basic Ultra Turrax® 0.0005-0.1 liter.

2. Preparation of Particles Using Dichloromethane

A 200 μl sample solution of 20 wt. % cetyl alcohol, 20 wt. % cetyl palmitate and 60 wt. % THC in dichloromethane was prepared by mixing 40 μl of stock solution 1), 40 μl of stock solution 2) and 120 μl of stock solution 3). The sample solution thus had a solute content of 10 mg/ml. An Ultra Turrax® vial was charged with 3.96 mL of water which was then subjected to stirring at 28.000 rpm. During stirring, 0.04 mL of the sample solution was added to the vial. After the addition, stirring was continued for 5 minutes. The resulting mixture was then collected in a syringe and filtered over a syringe filter (PTFE/PES/CA). The residue on the filter was washed with 4 mL of water by pushing the water through the filter. After reversal of the syringe filter, the product particles were removed from the filter and collected in a vial by pushing 4 mL of water through the filter into the vial. The water was then removed by evaporation to yield a dry, white solid. No yield was determined for this composition. Instead, the amount of isolated (encapsulated) THC was determined, and compared to the theoretical maximum yield of encapsulated THC of 0.40 mg (as is described in section 4 herebelow). Whereas compositions of THC are known to be tacky, this isolated product did not suffer from that. It was thus concluded that all THC present in the product was not present at an interface with air, and was thus encapsulated.

3. Preparation of Particles Using Benzyl Alcohol

The same procedure was followed as the one using dichloromethane, albeit with a few modifications. The sample solution was prepared from stock solutions 4), 5) and 6). Further, the dissolution of cetyl palmitate in benzyl alcohol was aided by warming the mixture to 55° C. A micrograph of the isolated product is displayed in FIG. 1, which forms direct evidence for the presence of the product of the invention in the form of particles. The photographed particles are of a spherical form while their surface appears to have some relief. Their diameter is in the range of 10-20 μm.

4. Encapsulation Efficiency

The amount of THC that ends up in the isolated product was compared to the theoretical maximum value of 0.40 mg (encapsulation efficiency). This was performed for the product obtained with benzyl alcohol. To this end, the solid product obtained under section 3 hereabove was dissolved in acetonitrile and the resulting solution was quantitatively analyzed using Gas Chromatography. This was performed in triplicate. The encapsulation efficiency was then calculated by dividing the measured amount of THC in the final dry and solid product by the theoretical maximum. The encapsulation efficiency was determined to be 85% for the product obtained with benzyl alcohol (based on GC analysis and HPLC studies performed in triplicate). It should be stressed that this value concerns all the stages from the initial mixing of the stock solutions, via the Ultra Turrax® stirring en syringe filter washing, to the final drying of the product.

5. Particle Composition

The quantitative analysis of the particles as described in section 4 with Gas Chromatography also yielded the amounts of cetyl alcohol and cetyl palmitate in the particles. Accordingly, the weight percentage of THC in the product (and thus in the particles) could be calculated. The obtained values averaged to 71 wt. % of THC.

6. Cannabinoid Stability Experiments

The stability of the cannabinoid contained in the particles prepared by the method of the invention was investgated by storing different samples of particles prepared as described in sections 2 and 3 hereabove, and analyzing them according to the procedure of section 4 hereabove. To this end, the samples were stored in a closed and dark container under atmospheric conditions. It appeared that no THC deterioration or decrease of the THC quantity could be observed with GC and HPLC during a period of as much as 13 months.

1. Process for preparing cannabinoid-containing particles, comprising
providing a solution wherein the following components are dissolved in a solvent that has a water solubility in the range of 2-100 g/L at 25° C.:
a cannabis component comprising delta-9-tetrahydrocannabinol;
a shell-forming component comprising one or more compounds selected from the group of C10-C30 fatty alcohols, C10-C30 fatty acids and esters of C10-C30 fatty alcohols and C10-C30 fatty acids;
generating droplets of the solution in an aqueous medium and allowing the solvent to migrate from the droplets to the aqueous medium to thereby form solid particles wherein a shell of shell-forming component encapsulates the cannabis component.

2. Process according to claim 1, wherein at least 99 wt. % of the cannabis component consists of delta-9-tetrahydrocannabinol and one or more cannabinoids selected from the group of cannabidiol, cannabinol and cannabigerol.

3. Process according to claim 1, wherein at least 99 wt. % of the cannabis component consists of delta-9-tetrahydrocannabinol.

4. Process according to claim 1, wherein the cannabis component comprises a whole-plant cannabis extract.

5. Process according to claim 1, wherein the solvent is selected from the group of 1-butanol, n-butyl acetate, gamma-butyrolacton, chloroform, 1,2-dichloroethane, diethylene glycol, diethyl ether, diethoxyethane, di-isopropylether, dimethyl sulfoxide, ethyl acetate, methyl t-butyl ether, N-methyl-2-pyrrolidinone, nitromethane, 1-pentanol, 2-pentanol, 3-pentanol, 3-pentanone, benzaldehyde, prenol, o-cresol, m-cresol, and p-cresol.

6. Process according to claim 1, wherein the solvent is benzyl alcohol.

7. Process according to claim 1, wherein the solvent has a solubility in water that is in the range of 8-80 g/L at 25° C., in particular in the range of 10-40 g/L at 25° C.

8. Process according to claim 1, wherein the shell-forming component comprises cetyl alcohol and/or cetyl palmitate.

9. Process according to claim 1, wherein the weight ratio of cannabis component to shell-forming component in the solution is in the range of 0.25:0.75 to 0.95:0.05, in particular in the range of 0.55:0.45 to 0.75:0.25.

10. Cannabinoid-containing particle obtainable by a process of claim 1.

11. Cannabinoid-containing particle, wherein
one or more cannabinoids including delta-9-tetrahydrocannabinol are encapsulated by a shell of one or more compounds selected from the group of C10-C30 fatty alcohols, C10-C30 fatty acids and esters of C10-C30 fatty alcohols and C10-C30 fatty acids;
the content of cannabinoid in the particle is in the range of 25-95 wt. %.

12. Cannabinoid-containing particle according to claim 10, wherein the shell is substantially free of cannabinoid.

13. Cannabinoid-containing particle according to claim 10, wherein the shell is substantially free of delta-9-tetrahydrocannabinol.

14. Cannabinoid-containing particle according to claim 10, wherein the weight ratio of cannabinoid to shell is in the range of 0.50:0.50 to 0.90:0.10 or in the range of 0.60:0.40 to 0.95:0.05.

15. Cannabinoid-containing particle according to claim 10, wherein the particle has a diameter in the range of 100 nm-500 μm, in particular in the range of 1-50 μm.

16. Pharmaceutical composition comprising a cannabinoid-containing particle according to claim 10 and an excipient.

17. Cannabinoid-containing particle according to claim 10 or a pharmaceutical composition according to claim 16 for use as a medicament.

18. Cannabinoid-containing particle according to claim 10 or a pharmaceutical composition according to claim 16, for use in the treatment of allergies, inflammations, infections, asthma, arthritis, epilepsy, depression, migraine, psychotic behavior, bipolar disorders, anxiety disorder, drug dependency and withdrawal syndromes, glaucoma, AIDS wasting syndrome, neuropathic pain, spasticity associated with multiple sclerosis, fibromyalgia, (chemotherapy-induced) nausea, anorexia, multiple sclerosis, gastrointestinal motility disorders, irritable bowel syndrome, appetite disorders, cachexia, and cramps.

19. Method for treating a medical condition of a human or an animal, comprising administering to the human or animal an effective amount of a cannabinoid-containing particle according to claim 10 or a pharmaceutical composition, wherein the medical condition is selected from the group of allergies, inflammations, infections, asthma, arthritis, epilepsy, depression, migraine, psychotic behavior, bipolar disorders, anxiety disorder, drug dependency and withdrawal syndromes, glaucoma, AIDS wasting syndrome, neuropathic pain, spasticity associated with multiple sclerosis, fibromyalgia, (chemotherapy-induced) nausea, anorexia, multiple sclerosis, gastrointestinal motility disorders, irritable bowel syndrome, appetite disorders, cachexia, and cramps.
